(19) United States
(12) Patent Application Publication
(10) Pub. No.: US 2012/0150100 A1
(43) Pub. Date: Jun. 14, 2012

(54) Laminar-Flow-Emitting Device

(76) Inventor: Carlos Ruiz Lapuente, Barcelona (ES)

(21) Appl. No.: 13/391,004

(22) PCT Filed: Aug. 17, 2010
(86) PCT No.: PCT/ES10/70560

§ 371 Date: Feb. 17, 2012

(30) Foreign Application Priority Data
Aug. 18, 2009 (ES) P200901785

(51) Int. Cl. A61M 37/00

(52) U.S. Cl. 604/23

(57) ABSTRACT

The invention relates to a laminar-flow-emitting device that can be incorporated into an operating theatre in order to allow eyes to be operated on safely. The invention is characterised by the special arrangement of the laminar flow outlets, which flow is projected towards the patient such as to facilitate the protection of both eyes simultaneously through the differentiated projection of two flows.

### OBJECT OF THE INVENTION

This invention is a laminar flow-emitting device that can be incorporated into an operating theatre in order to allow patients' eyes to be operated on under safe conditions.

The invention is characterised by the special arrangement of the laminar flow outlets, whose flow is projected towards the patient in such a way that both eyes are protected simultaneously through the differentiated projection of two flows.

### BACKGROUND OF THE INVENTION

It is a well known fact that there is an advantage in being able to project a laminar flow of air onto a work area, such as an eye, because it protects the eye from infection by preventing particles from entering the zone onto which the flow is being projected.

U.S. Pat. No. 7,311,700 describes a device that projects air onto a patient in such a way that the eye that is being operated on is immersed in a laminar flow. The device comprises a main body from which a pipe leads off, which is aimed at the patient to project the flow.

This laminar flow drags away any particles that are generated during the operation and does not return to the spot where the operation is being performed in view of the lack of recirculating flows such as the ones that occur when there is any turbulence. As the flow is filtered and does not contain any impurities, the fact that particles outside the flow are prevented from entering it ensures that the area that is affected by the flow projection is protected.

However, in the case of U.S. Pat. No. 7,311,700, projection by means of one single projection of laminar flow, ensures that the eye to be operated on is protected, but does not guarantee protection for the other eye. Furthermore, particles emitted during the operation could enter the other eye and cause it to be infected subsequently.

This invention manages to overcome the aforementioned technical problem by simultaneously protecting both eyes, because it establishes a differentiated flow that allows for both eyes to be protected; and through the different ways of realising it, the invention can solve other additional technical problems.

### DESCRIPTION OF THE INVENTION

This invention relates to a laminar flow-emitting device that permits the emission of a laminar flow that simultaneously reaches both the patient's eyes thus protecting not only the eye that is being operated on but also the other one.

This device comprises a main body containing at least one air impulse turbine, an element for filtering said air and elements for projecting the air flow under laminar conditions.

Where these elements are located is not relevant to the invention, although the most suitable place is to house them in the casing that constitutes the main body.

The filtering element removes particles that could affect the operating zone.

The elements that enable the flow to be emitted in laminar conditions are generally panels in the form of a honeycomb grill or arranged as tubular packages.

The invention is characterised in that the air flows out through two differentiated flows each of which is to be projected onto one or other of the patient's eyes.

Thus, with one single piece of equipment, when one eye is being operated on, the other eye is under the effects of a second flow that protects it from particles that come from the operating zone or elsewhere. This second protection makes it possible to reduce the risk of infection to which the eye not being operated on is exposed.

The embodiments of dependent claims 2 to 9 are also herein incorporated in their entirety by reference thereto.

### DESCRIPTION OF THE DRAWINGS

This descriptive memory is complemented by a set of drawings, with are illustrative of the preferred embodiment and by no means limitative of the invention.

FIG. 1 shows a perspective of an embodiment example of the invention shown in perspective.

FIG. 2 shows a detail of the head where the nozzles that project the laminar air are located. The head is partially sectioned so that the internal structure can be seen.

FIG. 3 is a plan view showing the layout of the device in an embodiment example of the invention together with a patient, so that a description can be given of the two differentiated flows making use of the device.

### DETAILED DISCLOSURE OF THE INVENTION

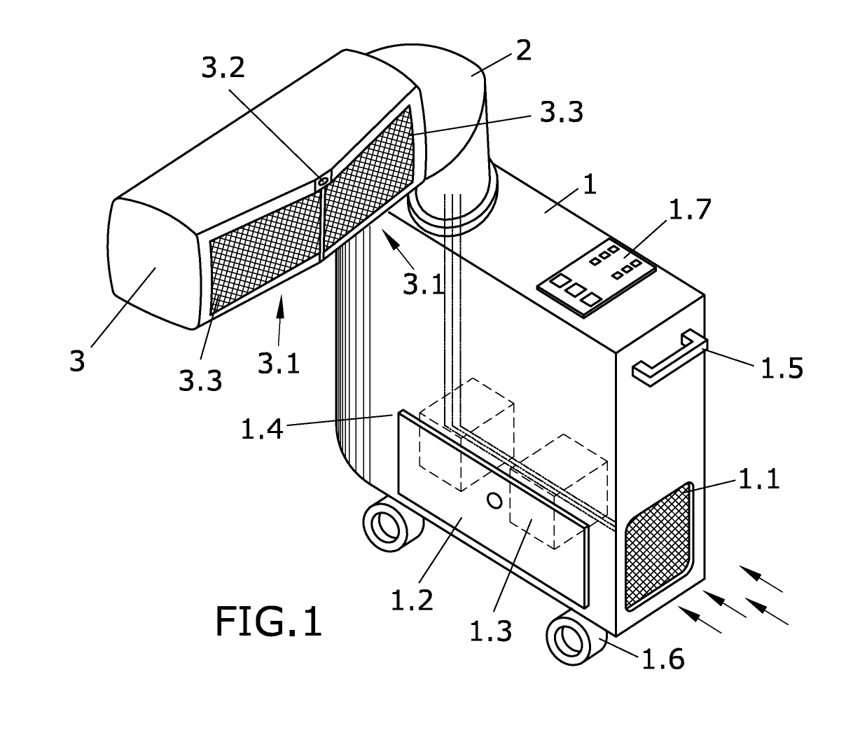

FIG. 1 shows a general perspective of an embodiment example of the invention where a main body (1) can be seen comprising a casing.

The main elements that act upon the impelled air are housed inside this main body (1); that is to say, a turbine (1.3), a filter (1.4), where in this example of the embodiment a HEPA H14 filter has been chosen because it is highly suitable, together with the pipes that convey the air from an air intake (1.1) to an outflow pipe (2).

A cover (1.2) can also be seen in this figure, providing access to the inside of the casing that forms the main body (1), and therefore for access to the internal components described.

The invention can be realised by changing the order of some of the components, such as the filter (1.4) and the impulse turbine (1.3) without this affecting the invention.

The same figure shows how air enters from the exterior through an air intake (1.1) that is equipped with a pre-filter for the largest particles. It is the HEPA filter that removes the finest particles from the flow.

The device is controlled by a set of controls (1.7) that in this embodiment example are on the top of the casing. This set of controls (1.7) is easily accessible to the physician who is performing the operation. It is this physician who has control over the flow conditions that are applied to the patient, such as the intensity of the flow or how long it is applied for.

In this embodiment example, the air driven by the turbine (1.3) flows out through an elbowed tubular outlet pipe (2) that has two first degrees of freedom, a first degree of freedom that corresponds to a turn with respect to the vertical axis, and a second degree of freedom that corresponds to the raising capacity if there is a vertical movement.

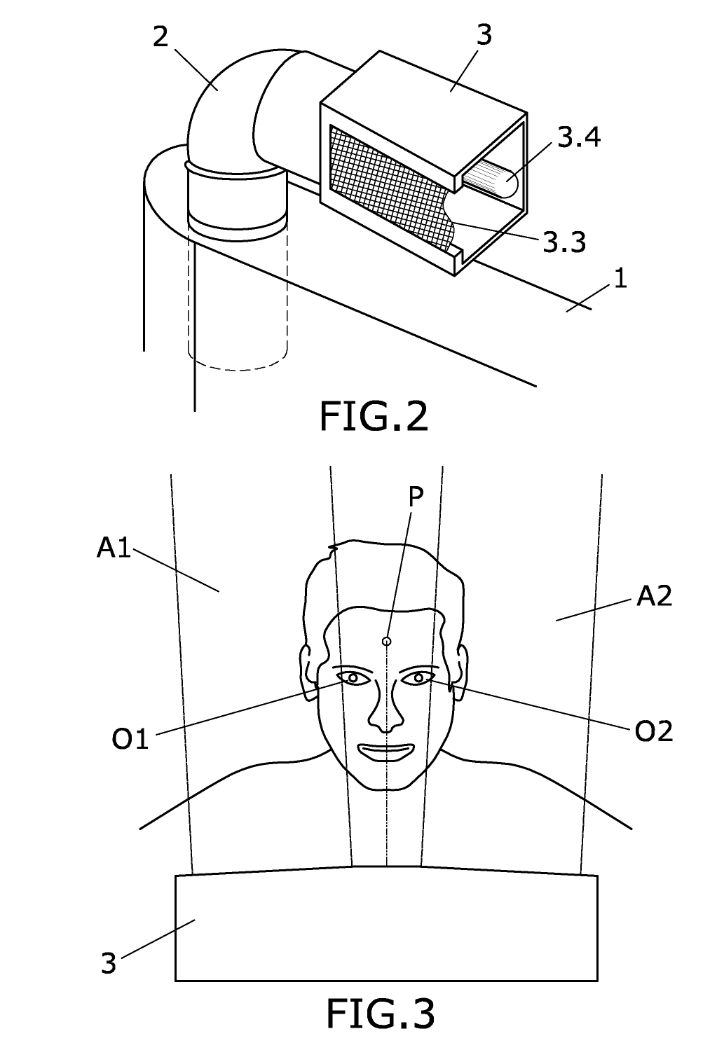

It can be seen in FIG. 2 that the elbowed tubular body that serves as the outlet pipe (2) also has a third degree of freedom that enables the user to modify the direction by a pitching movement of the elbowed end. This third degree of freedom consists of a turn with respect to a horizontal axis. The combination of the three degrees of freedom makes it possible to point the end of the outlet pipe (2) in any direction, and specifically to point in any direction, the elements that are installed on its end, such as the outlet head (3).

Therefore, in this embodiment example, the elbowed tubular body of the outlet pipe (2) ends at the outlet head (3). The position of the outlet head (3) and the direction in which it is facing thus determine the position and direction of the laminar outflows. All these operations can be motorised and controlled from the control means (1.7).

It is this end outlet head (3) that in this embodiment example enables two laminar flows to be emitted with distinct directions of emission. This emission with distinct directions takes place by incorporating two nozzles (3.1) on the same head (3). Each nozzle (3.1) consists of an outlet grille equipped with a laminar filter (3.3) to remove turbulence that occurs on a plane oriented according to a normal vector that is different for each nozzle (3.1); in this embodiment example, the normal vectors associated with each of the nozzles (3.1) are divergent. Thus, each nozzle (3.1) is aimed so that it can project a flow of laminar air into each of the patient's eyes.

A laser emitter (3.2) located between the two nozzles (3.1) is shown in this same embodiment example. This laser emitter (3.2) enables the outlet head (3.2) to be oriented to predict the points that the laminar flows of air emitted by the nozzles (3.1) will affect, even if the turbine (1.3) incorporated on the device is not operating.

It is in FIG. 2 that the partially sectioned outlet head (3) is shown, so that the internal structure and the presence of a source of light (3.4) can be seen. The source of light (3.4) emits light that can pass through the laminar filter (3.3), lighting up the zone where the laminar flow takes place, that is to say, the work zone. The work is thus made easier because there is more light. The set of controls (1.7) can be used to connect or disconnect the emission of light.

In operating theatres that are not equipped with air-flow-emitting devices in laminar conditions, the invention device can be used with the aid of a portable element on wheels (1.6) that can be gripped and moved because it has one or more handles (1.5).

FIG. 3 is a diagram that shows a plan view of the position of the patient (P) in an operating theatre. This operating theatre will be equipped with instruments, supports and other components that make it difficult to install other devices. FIG. 3 also shows the incorporation of the device according to the embodiment example already described, positioned on one side of the patient (P) and with the outlet head (3) facing his face.

This orientation of the outlet head (3) can be assisted by the laser emitter (3.2) in such a way that the outlet head (3) will be oriented, for example, until the point where the laser light is shining is on the forehead of the patient (P). In these conditions and because of the divergence of the mutual orientation between the outlet nozzles (3.1), each nozzle (3.1) will project its laminar air-flow (A1, A2) onto a different one of the patient's eyes (O1, O2). Similarly, if the physician who is performing the operation activates the light that comes from the internal source of light (3.4), the work zone, whichever eye (O1, O2) it happens to be, will also be lit up. This lighting is optional, because there are cases in which the light will cause reflections and it is advisable to have control over the way the work zone is lit up.

1. A laminar flow-emitting device that consists of a main body containing at least one impulse turbine, means for filtering the air and means for projecting the air flow in laminar conditions, wherein the air outflow takes place through two differentiated flows each of which is aimed at a different one of the same patient's eyes.

2. A device according to claim 1, wherein the two differentiated outflows are emitted from one single outlet head.

3. A device according to claim 2 wherein the differentiated flows are emitted from two nozzles located on the outlet head, where each one of the nozzles is facing in a different direction.

4. A device according to claim 2, wherein the outlet head can be oriented independently from the main body.

5. A device according to claim 2 wherein the outlet head is equipped with a laser emitter that makes it possible to help with the orientation of the head, depending on where the user wishes the air flows to be directed.

6. A device according to claim 5, wherein the orientation can be carried out in different ways by means of at least 2 degrees of freedom:
by turning according to a vertical axis, and
by a vertical movement.

7. A device according to claim 6, wherein additionally, the outlet head also has a third degree of freedom, comprising turning around a horizontal axis that enables it to be oriented by a pitching movement.

8. A device according to claim 1 wherein the outlet head is equipped with a source of light that is emitted from the air outlet to facilitate the lighting in the work zone.

9. A device according to claim 1, wherein the device is portable and equipped with wheels so the device can be moved.
